(19) United States
(12) Patent Application Publication
(10) Pub. No.: US 2019/0240167 A1
(43) Pub. Date: Aug. 8, 2019

(54) TWO-LAYER TOPICAL THERAPEUTIC SYSTEM

(71) Applicant: LTS Lohmann Therapie-Systeme AG, Andernach (DE)

(72) Inventors: Jessica VERSTRAELEN, Miesenheim (DE); Horst DZEKAN, Meinborn (DE); Nico REUM, Mendig (DE)

(21) Appl. No.: 16/343,195

(22) PCT Filed: Oct. 17, 2017
(86) PCT No.: PCT/EP2017/076442

§ 371 Date: Apr. 18, 2019

(30) Foreign Application Priority Data
Oct. 18, 2016 (EP) 16194418.6

(51) Int. Cl. A61K 9/70; A61K 31/196; A61K 31/201; A61K 31/355; A61K 47/32; A61P 29/00

(52) U.S. Cl. CPC A61K 9/7084; A61K 31/196; A61P 29/00; A61K 31/355; A61K 47/32; A61K 31/201

(57) ABSTRACT

There is described a topical therapeutic system, comprising a backing layer, a matrix layer containing active ingredient which contains at least one active ingredient, at least one permeability enhancer, at least one non-occlusive adhesive, at least one antioxidant, and at least one cross-linking agent, and also an occlusion-producing layer containing at least one adhesive, which layer is located between the matrix layer containing active ingredient and the backing layer.

### BACKGROUND

The present invention relates to a topical therapeutic system or to a topical patch containing active ingredient, especially with diclofenac (2-[2-[(2,6-dichlorophenyl)amino]phenyl]acetic acid) as active ingredient. The invention furthermore relates to the use of such a system as a medicament, especially in the therapy of pain and inflammatory conditions.

Topical therapeutic systems are a dosage form for administering drugs in patch form. These systems have certain advantages over conventional dosage forms. For example, sticking on the patch containing active ingredient means that the active ingredient is resorbed in the precise area and in the precise dose at the body site in question via the skin, without being prematurely broken down in the gastrointestinal tract or the liver. In addition, this dosage form makes it possible to dispense the active ingredient constantly over a longer period.

The administration of active ingredients is in many cases made more difficult by the low permeability of the skin. Therefore it was important to increase the penetrability of the skin for efficient resorption of active ingredient. One possible way of doing this is the effect of occlusion, which is understood to mean a build-up of water vapour in the upper dermal layers, which brings about greater permeability of the skin in relation to the active ingredient.

The occlusion of the patch is effected in accordance with DE 10103860 A1 by using a backing layer which is impermeable to water vapour, such as a thin plastics material film, preferably a polyethylene terephthalate (PET) film.

These known patches however have the disadvantage that as a rule they possess quite inelastic, rigid material properties, which results in lower wearing comfort, and with it restricted mobility of the wearer and frequent unintentional detaching of the patch.

One further possible way of increasing the permeability of the skin for absorbing an active ingredient is to use what are called penetration enhancers. EP 1 312 360 A1 discloses an analgesic, anti-inflammatory patch (“Dojin patch”) for local release of diclofenac. The system contains N-methyl-2-pyrrolidone as penetration enhancer and thus ensures increased permeation of the active ingredient via the skin. The disadvantage of this system is the health risk posed by this penetration enhancer. For according to ICH Guideline Q3C (of 4 Feb. 2011), a daily uptake of 5.3 mg N-methyl-2-pyrrolidone should not be exceeded.

### BRIEF DESCRIPTION

The aim of the present invention is to overcome the above-mentioned disadvantages of the prior art. Especially, the aim of the present invention is to provide a topical therapeutic system which brings about optimum resorption of an active ingredient via the skin, and does so without penetration enhancers which pose a health risk. Furthermore, this system should have high wearing comfort and strong adhesion, also to flexible body sites, such as joints.

The above aim is addressed by a topical therapeutic system which comprises a backing layer, a matrix layer containing active ingredient, which contains at least one active ingredient, at least one penetration enhancer, at least one non-occlusive adhesive, at least one antioxidant and at least one cross-linking agent, and also an occlusion-producing layer containing at least one adhesive, which layer is located between the matrix layer containing active ingredient and the backing layer.

### BRIEF DESCRIPTION OF THE DRAWINGS

FIG. 1 is a graphical illustration of in-vitro human skin permeation.

### DETAILED DESCRIPTION

“Occlusion” is understood here to mean the covering or closing, which is at least as extensive as possible, of regions of the skin with materials which are impermeable to water vapour. Consequently, hindering of insensible perspiration (water loss via the skin of a person at rest), hence a build-up of moisture and consequently hydration of the stratum corneum (outermost layer of the epidermis), occurs. Under occlusive conditions, the water content of the stratum corneum increases by up to 25% (m/m), preferably by up to 50% (m/m). The surface temperature of the skin also can increase to 37° C.

The at least one penetration enhancer is a compound which stabilises the active ingredient in dissolved form and thus ensures a relatively high resorption, which is also stable over a longer period, of the active ingredient via the skin.

A non-occlusive adhesive is to be understood to mean an adhesive which fixes the patch containing active ingredient as securely as possible to the skin, but without preventing insensible perspiration, i.e. the emergence of water vapour from the skin. Furthermore, the non-occlusive adhesive should be able to absorb the active ingredient in dissolved form, and to prevent it from crystallising out as largely as possible.

It is preferred for the non-occlusive adhesive to be a pressure-sensitive adhesive.

The at least one antioxidant is a chemical compound which prevents or reduces undesirable oxidation of other substances, specifically the active ingredient, and thus counteracts ageing of the therapeutic system. Specifically, antioxidants are distinguished by their action as free-radical scavengers and by the fact that they prevent the oxidative degradation of sensitive molecules, here specifically of the active ingredient contained, caused by atmospheric oxygen.

The at least one cross-linking agent is a chemical compound which ensures greater cohesion and greater strength of individual layers of the therapeutic system.

The topical therapeutic system according to the invention is constructed such that the matrix layer containing active ingredient lies on the skin. On that side of the matrix layer which does not touch the skin there is located the layer producing the occlusion, and on this there is the backing layer, so that the occlusion-producing layer is located between the matrix layer containing active ingredient and the backing layer.

The topical therapeutic system according to the invention is preferably characterised in that the backing layer comprises an elastic woven fabric, knitted fabric or non-woven fabric. This is extensible preferably in one, especially preferably in two, direction(s). This is understood to mean the extensibility or elasticity in the longitudinal and transverse direction, but not in the direction of the thickness, of the woven fabric, knitted fabric or non-woven fabric.

“Elasticity” or “extensible in both directions” is to be understood here to mean the ability of the topical therapeutic system to extend in two different directions, preferably in the longitudinal and transverse direction but not in the direction of the thickness, relative to the initial state of the material, without the basic shape being lost. Permanent deformation of the extended material does not occur. The elasticity is assessed with the aid of the extension, which is given as a dimensionless number or multiplied by 100 as a percentage. In the topical therapeutic system according to the invention, the extension in two different directions, preferably in the longitudinal direction and the transverse direction, is preferably 0 to 100%, especially preferably 10 to 50% and very especially preferably 15 to 30%, relative to the original dimensions of the topical therapeutic system. The elasticity is determined to ISO 13934-1 of 10 Apr. 2013.

The use of a non-occlusive woven fabric, knitted fabric or non-woven fabric which is extensible in both directions as backing layer is especially preferred.

The use of a woven fabric, knitted fabric or non-woven fabric which is extensible in both directions has the advantage that the topical therapeutic system according to the invention or the patch containing active ingredient, also in large embodiments and when stuck to flexible regions of the body, such as joints of the extremities, has high wearing comfort, no restriction of mobility and strong adhesion to the skin, and thus unintentional detaching is prevented.

Individual or combinations of active ingredient(s), preferably those with pain-relieving and inflammation-relieving action, may be contained as active ingredient in the topical therapeutic system according to the invention.

In a preferred embodiment, the topical therapeutic system according to the invention is characterised in that the active ingredient comprises at least one non-steroidal anti-inflammatory (NSAID). These non-steroidal anti-inflammatories (NSAIDs) are also known by the terms non-steroidal antirheumatics (NSARs) or alternatively non-steroidal antiphlogistics (NSAPs).

Examples of such non-steroidal anti-inflammatories are anthranilic acid derivatives (fenamates), such as mefenamic acid (Ponstan®), flufenamic acid (Assan®), etofenamate (Rheumon®, Traumalix®), meclofenamic acid (Meclomen®) and niflumic acid, Cox-2 inhibitors, such as celecoxib (Celebrex®), etoricoxib (Arcoxia®), acetic acid derivatives and arylacetic acid derivatives, such as aceclofenac (Beofenac®, D), acemetacin (Tilur®), bufexamac (Parfenac®), diclofenac, diclofenac gel (Voltaren®), etodolac (Lodine®), indometacin (Indocid®), ketorolac (Acular®, Toradol®), bromfenac (Yellox®), oxicams, such as lornoxicam (Xefo®), meloxicam (Mobicox®), piroxicam (Felden®), piroxicam gel, tenoxicam (Tilcotil®), propionic acid derivatives, such as ibuprofen, ibuprofen lysinate, ibuprofen arginate, ibuprofen sodium, dexibuprofen (Seractil®), naproxen (Aleve®), ketoprofen (Fastum®), dexketoprofen (Ketesse®), flurbiprofen (Froben®), benoxaprofen, tiaprofenic acid, (Surgam®, D), salicylates, such as acetylsalicylic acid (Aspirin®), calcium carbasalate (Alcacyl® tablets, Alca C®), lysine acetylsalicylate (Alcacyl® powder, Aspegic®), salicylic acid and others, such as nabumetone (Balmox®) and/or nimesulide (Aulin®).

In a preferred embodiment, the topical therapeutic system according to the invention is characterised in that the at least one active ingredient comprises diclofenac or a pharmaceutically acceptable salt thereof.

In one very especially preferred embodiment, the topical therapeutic system according to the invention is characterised in that the pharmaceutically acceptable salt is diclofenac sodium salt.

The active ingredient, especially the diclofenac salt, especially preferably the diclofenac sodium salt, is present in the matrix layer containing active ingredient in an amount of from 0.1 to 20 wt. %, preferably of from 0.5 to 15 wt. %, and especially preferably of from 1 to 10 wt. %, relative to the total weight of the matrix layer containing active ingredient.

Furthermore, the topical therapeutic system according to the invention is characterised in that the at least one penetration enhancer is not pyrrolidones, especially is not N-methyl-2-pyrrolidone, sulphoxides, especially dimethyl sulphoxide (DMSO), formamides, especially dimethyl formamide (DMF), and/or 1-dodecylazacycloheptan-2-one or laurocapram (azone), and/or derivatives thereof. This has the advantage that the wearer of the topical therapeutic system according to the invention is not exposed to the substances which pose a health risk N-methyl-2-pyrrolidone, dimethyl sulphoxide (DMSO), dimethyl formamide (DMF) and/or 1-dodecylazacycloheptan-2-one or laurocapram (azone), and/or derivatives thereof, and thus can wear the topical therapeutic system according to the invention over a longer period as well.

Preferably non-toxic compounds or compounds which do not pose a health risk, preferably selected from the group of fatty acids or fatty acid esters, are suitable as penetration enhancers.

The topical therapeutic system according to the invention is therefore preferably characterised in that the at least one penetration enhancer is selected from fatty acids and/or fatty acid esters, such as pentanoic acid, hexanoic acid, octanoic acid, nonanoic acid, decanoic acid, lauric acid, myristic acid, palmitic acid, stearic acid, arachidic acid, behenic acid, tetracosanoic acid, isoverlic acid, neoheptonic acid, neonanonic acid, isostearic acid, oleic acid, palmitoleic acid, linolenic acid, vaccenic acid, petroselinic acid, elaidic acid, oleic acid, arachidonic acid, gadoleic acid, erucic acid, ethyl acetate, methyl propylate, butyl acetate, methyl valerate, diethyl sebacitate, methyl laurate, ethyl oleate, isopropyl decanoate, isopropyl myristate (myristic acid isopropyl ester), isopropyl palmitate, isopropyl oleinate (oleic acid isopropyl ester), preferably oleic acid, lauric acid and/or myristic acid, especially preferably oleic acid, and/or fatty acid esters, preferably oleic acid isopropyl ester and/or myristic acid isopropyl ester.

The at least one penetration enhancer is preferably present in an amount of from 1 to 50 wt. %, especially preferably in an amount of from 2 to 40 wt. % and very especially preferably in an amount of from 5 to 30 wt. %, relative to the total weight of the matrix layer containing active ingredient.

Using fatty acids and/or fatty acid esters as penetration enhancers, and especially dispensing with pyrrolidones, especially N-methyl-2-pyrrolidone, sulphoxides, especially dimethyl sulphoxide (DMSO), formamides, especially dimethyl formamide (DMF) and/or 1-dodecylazacycloheptan-2-one or laurocapram (azone), and/or derivatives thereof, yields the advantage that the user of the topical therapeutic system according to the invention is not exposed to any penetration enhancer which poses a health risk, which enables him to use the topical therapeutic system according to the invention over a longer period as well.

In order to stick the topical therapeutic system securely to the skin, it is preferable for the at least one non-occlusive adhesive to comprise a pressure-sensitive adhesive.

It is furthermore preferable for the at least one non-occlusive adhesive, preferably the at least one non-occlusive pressure-sensitive adhesive, to comprise an adhesive, preferably a pressure-sensitive adhesive, on the basis of an acrylate copolymer. The use of an acrylate/vinyl acetate copolymer is especially preferred.

Preferably the non-occlusive adhesive, preferably a pressure-sensitive adhesive, on the basis of an acrylate copolymer, preferably an acrylate/vinyl acetate copolymer, contains free hydroxyl groups, which ensure optimum solubility of the at least one active ingredient, preferably the diclofenac.

In a further preferred embodiment, the non-occlusive adhesive, a pressure-sensitive adhesive, preferably on the basis of an acrylate copolymer, especially preferably of an acrylate/vinyl acetate copolymer, is a non-acidic adhesive, i.e. an adhesive which does not contain any free carboxyl groups, which largely prevents the at least one active ingredient, preferably the diclofenac sodium salt, from crystallising out.

The non-occlusive adhesive, preferably a pressure-sensitive adhesive, is preferably contained in the matrix layer containing active ingredient in an amount of from 20 to 99 wt. %, especially preferably of from 40 to 99 wt. %, very especially preferably of from 50 to 98 wt. %, and especially of from 70 to 80 wt. %, relative to the total weight of the matrix layer containing active ingredient.

In one very especially preferred embodiment, the non-occlusive adhesive, preferably a pressure-sensitive adhesive, on the basis of an acrylate/vinyl acetate copolymer is “DuroTak 387-2287” (from Henkel, Germany).

In a further preferred embodiment, the topical therapeutic system according to the invention is characterised in that the at least one antioxidant is selected from alpha-tocopherol, ascorbyl palmitate and butylhydroxytoluene. The at least one antioxidant is preferably contained in the matrix layer containing active ingredient in an amount of from 0.001 to 5 wt. %, preferably of from 0.01 to 4 wt. %, and especially preferably of from 0.05 to 3 wt. %, relative to the total weight of the matrix layer containing active ingredient.

The use of an antioxidant has the advantage that the topical therapeutic system according to the invention remains stable over a longer period and under the most varied external conditions.

In a preferred embodiment, the topical therapeutic system according to the invention is stable over a period of 36 months in all three ICH climatic zones (25° C./60% relative humidity (RH), 30° C./65% RH and 30° C./75% RH).

Preferably the topical therapeutic system according to the invention is characterised in that the at least one cross-linking agent is selected from the group of metal chelates, preferably aluminium acetylacetonate.

Preferably the at least one cross-linking agent, which especially preferably is aluminium acetylacetonate, is contained in an amount of from 0.01 to 25 wt. %, preferably of from 0.05 to 20 wt. %, and especially preferably of from 0.1 to 10 wt. %, relative to the total weight of the matrix layer containing active ingredient.

The cross-linking agent causes the matrix layer containing active ingredient to be more uniform and stronger, i.e. to lead to an increase in the cohesion of the matrix layer containing active ingredient. Thus when detaching the patch it is ensured that the matrix layer containing active ingredient does not partly remain behind on the user's skin, but is detached completely.

The layer producing the occlusion preferably comprises an adhesive. This is preferably a polyisobutylene adhesive, especially preferably a polyisobutylene adhesive on the basis of a low-molecular polyisobutylene adhesive and of a high-molecular polyisobutylene adhesive. “Low-molecular” is understood here to mean a polyisobutylene adhesive with an average molecular weight of 5000 to 490000 g/mol, and “high-molecular” to mean a polyisobutylene adhesive with an average molecular weight of 500000 to 5 million g/mol. The molecular weight in this case is taken from the certificates of analysis of the respective manufacturer which were valid on the application date.

Preferably the low-molecular polyisobutylene adhesive is “Oppanol B 10 SFN”, and the high-molecular polyisobutylene adhesive is “Oppanol B 100” (both from BASF).

The mixture ratio of low-molecular polyisobutylene adhesive to high-molecular polyisobutylene adhesive is preferably about 60 to 90 to about 40 to 10 parts by weight, especially preferably about 70 to 90 to about 30 to 10 parts by weight, and very especially preferably about 85 to about 15 parts by weight.

Especially the high-molecular content of polyisobutylene adhesive is responsible for the occlusion of the system. Thus the occlusion can also be adapted by changing the mixture ratio.

The topical therapeutic system according to the invention is further preferably distinguished in that the matrix layer containing active ingredient has a basis weight of from 40 to 160 g/m², preferably of from 50 to 120 g/m², and especially preferably of from 60 to 100 g/m².

The layer producing the occlusion has a basis weight of from 5 to 150 g/m², preferably of from 10 to 120 g/m², and especially preferably of from 20 to 100 g/m².

Furthermore, the topical therapeutic system according to the invention may optionally contain a fragrance.

In one very especially preferred embodiment, the topical therapeutic system according to the invention is characterised in that the at least one active ingredient is diclofenac sodium salt, the at least one penetration enhancer is oleic acid, the at least one non-occlusive adhesive is an adhesive on the basis of an acrylate/vinyl acetate copolymer, the antioxidant is selected from alpha-tocopherol and/or ascorbyl palmitate, the at least one cross-linking agent is aluminium acetylacetonate and the layer producing the occlusion comprises an adhesive on the basis of a mixture of a low-molecular polyisobutylene adhesive and of a high-molecular polyisobutylene adhesive.

In a further preferred embodiment, the topical therapeutic system according to the invention is characterised in that it comprises a surface area of about 40 to 250 cm², preferably of about 70 and about 160 cm².

Further, the topical therapeutic system according to the invention in a preferred embodiment is characterised in that it comprises a removable protective layer, preferably a siliconised polyethylene terephthalate film, which is glued on to that side of the matrix layer containing active ingredient which is not the layer producing the occlusion. This removable protective layer makes the topical therapeutic system according to the invention able to be packed and transported. In the very especially preferred embodiment of the topical therapeutic system according to the invention described above, the term “comprise” may also mean “consisting of”.

Further, the topical therapeutic system according to the invention in a especially preferred embodiment is characterised in that there is contained in the matrix layer containing active ingredient the at least one active ingredient diclofenac sodium salt in an amount of from 1 to 10 wt. %, the at least one penetration enhancer oleic acid in an amount of from 5 to 30 wt. %, the at least one non-occlusive adhesive on the basis of an acrylate/vinyl acetate copolymer in an amount of from 50 to 98 wt. %, the antioxidant alpha-tocopherol in an amount of from 0.05 to 3 wt. %, and the at least one cross-linking agent aluminium acetylacetonate in an amount of from 0.1 to 10 wt. %, all relative to the matrix layer containing active ingredient, and the layer producing the occlusion comprises a polyisobutylene adhesive on the basis of a low-molecular polyisobutylene adhesive and of a high-molecular polyisobutylene adhesive.

The present invention also relates to a topical therapeutic system as described above as a medicament.

Further, the present invention relates to a topical therapeutic system as described above, for use in pain and inflammatory conditions, such as for example in inflammatory rheumatic diseases, such as chronic polyarthritis, fibromyalgia or arthrosis, acute attacks of gout, joint injuries in sport, pain and swelling after operations, prolapsed discs, venous diseases.

The invention will be discussed below with reference to non-limitative examples.

### Examples

1. Topical therapeutic systems A, B, C, D and E with the matrix layer containing active ingredient shown in Table 1, and a backing layer of PET, woven PBT fabric or non-woven fabric or knitted fabric or a PET film (system E) were produced as follows:

TABLE 1

| | | Non- | Cross- | | | | |
|---|---|---|---|---|---|---|---|
| | Active | occlusive | linking | | Fragrance | | |
| | ingredient | adhesive | agent | Penetration | Eucalyptus | Antioxidant | |
| Topical | Diclofenac- | DuroTak | Al acetyl- | enhancer | Sting | Alpha- | |
| therapeutic | Na | 387-2287 | acetonate | Oleic acid | (Novartis) | tocopherol | Occlusion |
| system | [%] | [%] | [%] | [%] | [%] | [%] | Effect due to |
| A^{(a)} | 1 | | | | | | SIS/PBM^{(b)} |
| | | | | | | | matrix |
| B | 4 | 78.8 | 1.2 | 15.0 | 0.5 | 0.5 | Occlusive layer |
| | | | | | | | between |
| | | | | | | | backing layer |
| | | | | | | | and matrix |
| | | | | | | | layer |
| | | | | | | | containing |
| | | | | | | | active |
| | | | | | | | ingredient: 30 g/m² |
| | | | | | | | PIB^{(c)} |
| C | 4 | 78.8 | 1.2 | 15.0 | 0.5 | 0.5 | Occlusive layer |
| | | | | | | | between |
| | | | | | | | backing layer |
| | | | | | | | and matrix |
| | | | | | | | layer |
| | | | | | | | containing |
| | | | | | | | active |
| | | | | | | | ingredient: 60 g/m² |
| | | | | | | | PIB^{(c)} |
| D | 4 | 95.0 | — | — | 0.5 | 0.5 | Occlusive layer |
| | | | | | | | between |
| | | | | | | | backing layer |
| | | | | | | | and matrix |
| | | | | | | | layer |
| | | | | | | | containing |
| | | | | | | | active |
| | | | | | | | ingredient: 30 g/m² |
| | | | | | | | PIB^{(c)} |
| E | 4 | 78.8 | 1.2 | 15.0 | 0.5 | 0.5 | PET^{(d)} film on |
| | | | | | | | the backing |
| | | | | | | | layer |

[%] Percent by weight, relative to the weight of the matrix layer containing active ingredient
^{(a)}Topical therapeutic system (“Dojin”) containing N-methyl-2-pyrrolidones as penetration enhancer, disclosed in EP 1 312 360 A1
^{(b)}Styrene-isoprene-styrene copolymer; polyisobutylene
^{(c)}Polyisobutylene adhesive (85 parts by weight Oppanol B 10 and 15 parts by weight Oppanol B 100)
^{(d)}Polyethylene terephthalate

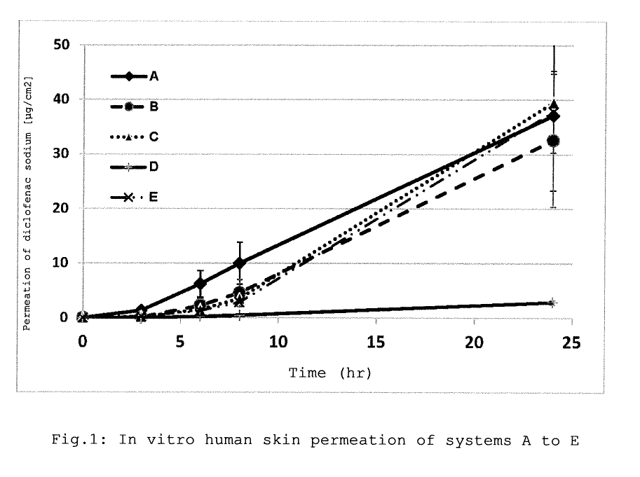

The in vitro human skin permeation of the systems listed in Example 1 was measured with the aid of a Franz cell. The substance or formulation (e.g. gels, ointments, solutions, patches) is located in the donor compartment. The acceptor compartment is filled with buffer or other solutions. The permeation of a substance over the selected period through the skin can be followed by regular sampling from the acceptor compartment. Likewise, the influence of penetration enhancers on the permeation of a substance can be tested using this system. The use of the Franz cell as a diffusion model is suitable above all for predicting the transport of drugs through human skin (=permeation), which corresponds to the systemic availability. It is important to note here that there is no in vitro-in vivo correlation. In this case, the Franz cell was loaded with human abdominal skin obtained from operations. In this case, 500 μm dermatomised skin with a diffusion area of 1.165 cm² was incubated with the topical therapeutic system. An aqueous isotonic phosphate buffer pH=7.4 plus 0.1% Na azide with a filling volume of 10 ml served as acceptor medium. The measurement of the permeation was carried out at a temperature of 32° C. and was measured after 3, 6, 8 and 24 hours (n=3), and can be inferred from FIG. 1.

Systems A, D and E are comparison examples.

System A was based on the following constituents: composition corresponding to EP 1 312 360 A1 (“Dojin patch”).

No penetration enhancer was used in system D. In the last column of Table 1 it is indicated how the occlusion was achieved. In system E, a PET film was applied to the backing layer. The backing layer is located directly on the matrix layer containing active ingredient (without an additional layer of adhesive).

TABLE 2
In vitro human skin permeation of systems A to E

| | Total diclofenac-Na | | | | | | | | | |
|---|---|---|---|---|---|---|---|---|---|---|
| | permeation in [μg/cm²] | | | | | Standard deviation | | | | |
| Sys- | Time [hr] | | | | | | | | | |
| tem | 0 | 3 | 6 | 8 | 24 | 0 | 3 | 6 | 8 | 24 |
| A | 0 | 1.35 | 6.21 | 9.99 | 37.10 | 0 | 0.63 | 2.41 | 3.83 | 13.7 |
| B | 0 | 0.11 | 2.25 | 4.58 | 32.58 | 0 | 0.14 | 1.30 | 2.44 | 12.17 |
| C | 0 | 0.23 | 1.50 | 3.50 | 39.50 | 0 | 0 | 0.52 | 0.89 | 3.93 |
| D | 0 | — | 0.21 | 0.44 | 2.88 | 0 | — | 0.05 | 0.09 | 0.38 |
| E | 0 | 0.25 | 1.27 | 2.85 | 37.80 | 0 | 0 | 0.60 | 0.86 | 7.5 |

FIG. 1 and Table 2 show the in vitro human skin permeation of systems A to E. The systems according to the invention B and C exhibit a permeation of the active ingredient of comparable quality to the known system A, but with oleic acid as penetration enhancer instead of N-methyl-2-pyrrolidone, which poses a health risk, in the comparison system.

Comparison of systems B and C with comparison system E shows comparable permeation of the active ingredient, to be attributed to comparable occlusion, but without using an occlusive support material in systems B and C.

Comparison of systems B and C with system D shows the influence of the penetration enhancer oleic acid on the resorption of the active ingredient.

In Table 3 below, the water-vapour transmissions of the formulations which were determined in a laboratory are given. Formulation F corresponds to formulation E, but without PET film. Diclofenac-Na patch formulations with values of less than 500 g/m² water in 24 hr are sufficient to achieve the permeation of diclofenac-Na through the skin in comparable orders of magnitude to the Dojin patch.

It is known that the amount of water emitted through the skin of the underarm of a human at rest is 120-168 g/m² in 24 hours (dissertation of Christiane Fauth, Dec. 12, 2003, Universitat Halle a.d. Saale). Very probably the value of the water loss of a human who is moving is significantly higher.

TABLE 3
Water-vapour transmissions of systems A to F

| | Water-vapour transmission [g/m²] | | | | | |
|---|---|---|---|---|---|---|
| System | After 24 hr | | After 48 hr | | After 72 hr | |
| A | 13.9 | ±4.35 | 22.5 | ±5.54 | 21.3 | ±4.28 |
| B | 180.2 | ±57.56 | 371.9 | ±101.68 | 659.5 | ±43.56 |
| C | 4.9 | ±2.31 | 27.9 | ±0.83 | 66.2 | ±17.73 |
| D | 26.5 | ±2.17 | 33.0 | ±3.93 | — | — |
| E | 319.9 | ±19.49 | 313.3 | ±16.64 | 321.0 | ±12.18 |
| F | 4192.7 | ±149.86 | 4171.6 | ±118.69 | 4219.5 | ±124.45 |

The exemplary embodiment has been described with reference to the preferred embodiments. Obviously, modifications and alterations will occur to others upon reading and understanding the preceding detailed description. It is intended that the exemplary embodiment be construed as including all such modifications and alterations insofar as they come within the scope of the appended claims or the equivalents thereof.

1. A topical therapeutic system, comprising:
a backing layer,
a matrix layer containing active ingredient, which contains
at least one active ingredient,
at least one penetration enhancer,
at least one non-occlusive adhesive on the basis of an acrylate/vinyl acetate
copolymer which contains free hydroxyl groups,
at least one antioxidant, and
at least one cross-linking agent,
and also an occlusion-producing layer containing at least one adhesive, which layer is located between the matrix layer containing active ingredient and the backing layer, wherein the at least one active ingredient comprises diclofenac or a pharmaceutically acceptable salt thereof.

2. A topical therapeutic system according to claim 1, characterised in that the backing layer contains an elastic woven fabric, knitted fabric or non-woven fabric.

3. A topical therapeutic system according to claim 1, characterised in that the at least one active ingredient comprises diclofenac sodium salt.

4. A topical therapeutic system according to claim 1, characterised in that the at least one penetration enhancer is not N-methyl-2-pyrrolidone.

5. A topical therapeutic system according to claim 1, characterised in that the at least one penetration enhancer is selected from fatty acids and/or fatty acid esters.

6. A topical therapeutic system according to claim 1, characterised in that the at least one penetration enhancer is present in the matrix layer containing active ingredient in an amount of from 1 to 50 wt. %, relative to the weight of the matrix layer containing active ingredient.

7. A topical therapeutic system according to claim 1, characterised in that the at least one antioxidant is selected from alpha-tocopherol, ascorbyl palmitate and butylhydroxytoluene.

8. A topical therapeutic system according to claim 1, characterised in that the at least one cross-linking agent is selected from the group of metal chelates.

9. A topical therapeutic system according to claim 1, characterised in that the layer producing the occlusion comprises a polyisobutylene adhesive.

10. A topical therapeutic system according to claim 1, characterised in that the at least one active ingredient is diclofenac sodium salt, the at least one penetration enhancer is oleic acid, the at least one non-occlusive adhesive is an adhesive on the basis of an acrylate/vinyl acetate copolymer which contains free hydroxyl groups, the antioxidant is alpha-tocopherol, the at least one cross-linking agent is aluminium acetylacetonate and the layer producing the occlusion comprises a polyisobutylene adhesive on the basis of a low-molecular polyisobutylene adhesive and of a high-molecular polyisobutylene adhesive.

11. A topical therapeutic system according to claim 10, characterised in that there is contained in the matrix layer containing active ingredient the at least one active ingredient diclofenac sodium salt in an amount of from 1 to 10 wt. %, the at least one penetration enhancer oleic acid in an amount of from 5 to 30 wt. %, the at least one non-occlusive adhesive on the basis of an acrylate/vinyl acetate copolymer which contains free hydroxyl groups in an amount of from 50 to 98 wt. %, the antioxidant alpha-tocopherol in an amount of from 0.05 to 3 wt. %, and the at least one cross-linking agent aluminium acetylacetonate in an amount of from 0.1 to 10 wt. %, all relative to the matrix layer containing active ingredient, and the layer producing the occlusion comprises a polyisobutylene adhesive on the basis of a low-molecular polyisobutylene adhesive and of a high-molecular polyisobutylene adhesive.

12. A method of administering a medicament to a patient comprising applying the topical therapeutic system according to claim 1 to a skin of the patient.

13. A topical therapeutic system according to claim 5, wherein the at least one penetration enhancer comprises oleic acid, myristic acid and/or lauric acid or esters thereof.

14. A topical therapeutic system according to claim 13, wherein the at least one penetration enhancer comprises myristic acid isopropyl ester and/or oleic acid isopropyl ester.

15. A topical therapeutic system according to claim 8, wherein the at least one cross-linking agent comprises aluminium acetylacetonate.

16. A topical therapeutic system according to claim 9, wherein the polyisobutylene adhesive comprises a low-molecular weight polyisobutylene adhesive and a high-molecular weight polyisobutylene adhesive.

17. A method of treating pain or inflammation in a patient comprising the topical application of a therapeutic system, said system comprising a backing layer, a matrix layer containing active ingredient, which contains at least one active ingredient, at least one penetration enhancer, at least one non-occlusive adhesive on the basis of an acrylate/vinyl acetate copolymer which contains free hydroxyl groups, at least one antioxidant, and at least one cross-linking agent, and also an occlusion-producing layer containing at least one adhesive, which layer is located between the matrix layer containing active ingredient and the backing layer, wherein the at least one active ingredient comprises diclofenac or a pharmaceutically acceptable salt thereof.
